(19) United States
(12) Patent Application Publication
(10) Pub. No.: US 2024/0006017 A1
(43) Pub. Date: Jan. 4, 2024

(54) Protein Structure Prediction

(71) Applicant: Microsoft Technology Licensing, LLC, Redmond, WA (US)

(72) Inventors: Tong Wang, Redmond, WA (US); Bin Shao, Beijing (CN); Tie-Yan Liu, Beijing (CN)

(21) Appl. No.: 18/037,682

(22) PCT Filed: Dec. 8, 2021
(86) PCT No.: PCT/US2021/062292

§ 371 Date: May 18, 2023

(30) Foreign Application Priority Data
Dec. 31, 2020 (CN) 202011623825.0

(51) Int. Cl. G16B 15/20; G16B 40/20

(52) U.S. Cl. CPC G16B 15/20; G16B 40/20

(57) ABSTRACT

According to implementations of the subject matter described herein, there is provided a solution for protein structure prediction. In this solution, a constraint set for a target protein is obtained, the constraint set comprising constraints for structural properties of the target protein. Feature information is extracted from the constraints respectively, and weights corresponding to the constraints are determined respectively based on the feature information of the constraints. Each weight indicates a degree of influence of the corresponding constraint in prediction of a structure of the target protein. The structure of the target protein is predicted based on the constraints in the constraint set and the weights. According to the solution, through the pre-processing on the constraints for use, it is possible to solve potential conflicts in the constraint set and eliminate constraint redundancy. This enables accurate prediction of the structure of the target protein.

### BACKGROUND

Proteins are biomolecules or macromolecules composed of long chains of amino acid residues. Proteins perform many significant life activities in organisms, and functions of the proteins are mainly determined by their three-dimensional (3D) structures. Knowing the structures of proteins enables understanding the functions of proteins, interaction between proteins, how proteins perform their biological functions, and so on. This is very important to the fields of medicine and biotechnology. For example, if a certain protein plays a key role in a disease, drug molecules can be designed based on the structure of the protein to treat the disease.

Currently, the structures of the proteins are generally studied through experiments. However, it is quite time-consuming to determine the structures of proteins through the experiments. As compared with the number of proteins existing in nature, only a small number of proteins whose structures are determined through experiments. Therefore, it has become an important means in protein structure research to predict protein structures at a low cost and with a high yield.

### SUMMARY

According to implementations of the subject matter described herein, there is provided a solution for protein structure prediction. In this solution, a constraint set for a target protein is obtained, the constraint set comprising a plurality of constraints for a plurality of structural properties of the target protein. Feature information is extracted from the plurality of constraints respectively, and a plurality of weights corresponding to the plurality of constraints are determined respectively based on the feature information of the plurality of constraints. Each weight indicates a degree of influence of the corresponding constraint in prediction of a structure of the target protein. The structure of the target protein is predicted based on the plurality of constraints in the constraint set and the plurality of weights. According to the solution, through the pre-processing on the constraints for use, it is possible to solve potential conflicts in the constraint set and eliminate constraint redundancy. This enables accurate prediction of the structure of the target protein.

The Summary is to introduce a selection of concepts in a reduced form that are further described below in the Detailed Description. The Summary is not intended to identify key features or essential features of the subject matter described herein, nor is it intended to be used to limit the scope of the subject matter described herein.

### BRIEF DESCRIPTION OF THE DRAWINGS

FIG. 1 illustrates a block diagram of a computing device which can implement various implementations of the subject matter described herein;

FIG. 2 illustrates a schematic diagram of structural properties of a protein;

FIG. 3 illustrates a schematic diagram of an example spatial coordinate representation system of an atom of a protein;

FIG. 4 illustrates a block diagram of a protein structure prediction system according to some implementations of the subject matter described herein;

FIG. 5A and FIG. 5B illustrate two examples of constraints for structural properties according to some implementations of the subject matter described herein;

FIG. 6 illustrates a block diagram of a protein structure prediction system according to some other implementations of the subject matter described herein;

FIG. 7 illustrates a block diagram of a protein structure prediction system according to some other implementations of the subject matter described herein;

FIG. 8 illustrates an example comparison of conflicts and redundancy between constraints in the constraint set before and after iterative filtration according to some implementations of the subject matter described herein;

FIG. 9 illustrates an example comparison of iterative protein structure prediction with and without genetic initialization according to some implementations of the subject matter described herein;

FIG. 10 illustrates a flowchart of a process of protein structure prediction according to some implementations of the subject matter described herein.

Throughout the drawings, the same or similar reference symbols refer to the same or similar elements.

### DETAILED DESCRIPTION OF EMBODIMENTS

Principles of the subject matter described herein will now be described with reference to some example implementations. It is to be understood that these implementations are described only for the purpose of illustration and help those skilled in the art to better understand and thus implement the subject matter described herein, without suggesting any limitations to the scope of the subject matter disclosed herein.

As used herein, the term “includes” and its variants are to be read as open terms that mean “includes, but is not limited to.” The term “based on” is to be read as “based at least in part on.” The terms “an implementation” and “one implementation” are to be read as “at least one implementation.” The term “another implementation” is to be read as “at least one other implementation.” The term “first,” “second,” and the like may refer to different or the same objects. Other definitions, either explicit or implicit, may be included below.

As used herein, the term “model” is referred to as an association between an input and an output learned from training data, and thus a corresponding output may be generated for a given input after the training. The generation of the model may be based on a machine learning technique. Deep learning is one of the machine learning algorithms which processes an input and provide the corresponding output using processing units in multiple layers. Neural network model is an example deep learning model. As used herein, “model” may also be referred to as “machine learning model”, “learning model”, “machine learning network”, or “learning network,” which are used interchangeably herein.

“Neural network” is a machine learning network based on deep learning. A neural network can process an input and provides a corresponding output and it generally includes an input layer, an output layer and one or more hidden layers between the input and output layers. The neural network used in the deep learning applications generally includes a plurality of hidden layers to increase the depth of the network. Individual layers of the neural network model are connected in sequence, such that an output of a preceding layer is provided as an input for a following layer, where the input layer receives the input of the neural network while the output of the output layer acts as the final output of the neural network. Each layer of the neural network includes one or more nodes (also referred to as processing nodes or neurons), each of which processes the input from the preceding layer.

Generally, machine learning may include three stages, i.e., a training stage, a test stage, and an application stage (also referred to as an inference stage). In the training stage, a given machine learning network may be trained iteratively using a great amount of training data until the network can obtain, from the training data, consistent inference similar to those that human intelligence can make. Through the training, the machine learning network may be regarded as being capable of learning the association between the input and the output (also referred to an input-output mapping) from the training data. The set of parameter values of the trained network is determined. In the test stage, a test input is applied to the trained model to test whether the machine learning network can provide a correct output, so as to determine the performance of the network. In the application stage, the machine learning network may be used to process an actual network input based on the set of parameter values obtained in the training and to determine the corresponding network output.

The structure of a protein is usually divided into a plurality of levels, including a primary structure, a secondary structure, a tertiary structure and so on. The primary structure refers to the arrangement order of amino acids, i.e., an amino acid sequence. The secondary structure refers to a specific conformation formed by main chain atoms along a certain axis, which includes, but is not limited to, α-helix, β-fold, coil, and so on. The tertiary structure refers to a three-dimensional (3D) spatial structure formed through further coiling and folding of the protein on the basis of the secondary structure. A protein fragment (also referred to as a “fragment” for short) comprises a plurality of amino acid residues arranged in a three-dimensional spatial structure. A peptide is a protein fragment which includes two or more amino acids connected via peptide bonds.

As mentioned above, the structure of a protein mainly affects its functionality, and protein structure prediction, especially the prediction of the tertiary structure becomes the important means in protein structure research.

### Example Environment

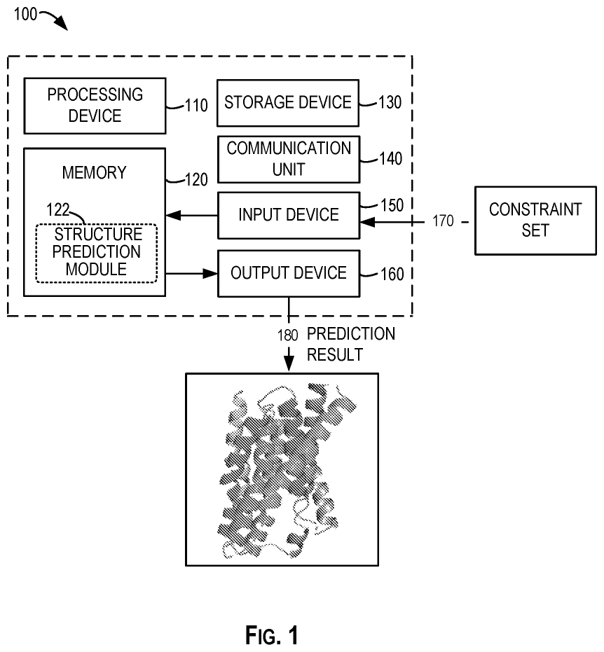

FIG. 1 illustrates a block diagram of a computing device 100 which can implement various implementations of the subject matter described herein. It should be understood that the computing device 100 shown in FIG. 1 is only exemplary and should not suggest any limitation on the functions and scopes of the implementations described by the subject matter described herein. As shown in FIG. 1, the computing device 100 includes a computing device 100 in the form of a general purpose computing device. Components of the computing device 100 may include, but is not limited to, one or more processors or processing units 110, a memory 120, a storage device 130, one or more communication units 140, one or more input devices 150, and one or more output devices 160.

In some implementations, the computing device 100 may be implemented as various user terminals or service terminals with computing capability. The service terminals may be servers, large-scale computing devices, and the like provided by a variety of service providers. The user terminal, for example, is a mobile terminal, a fixed terminal or a portable terminal of any type, including a mobile phone, a site, a unit, a device, a multimedia computer, a multimedia tablet, Internet nodes, a communicator, a desktop computer, a laptop computer, a notebook computer, a netbook computer, a tablet computer, a Personal Communication System (PCS) device, a personal navigation device, a Personal Digital Assistant (PDA), an audio/video player, a digital camera/video, a positioning device, a television receiver, a radio broadcast receiver, an electronic book device, a gaming device or any other combination thereof, including accessories and peripherals of these devices or any other combination thereof. It may also be predicted that the computing device 100 can support any type of user-specific interface (such as a “wearable” circuit, and the like).

The processing unit 110 may be a physical or virtual processor and may execute various processing based on the programs stored in the memory 120. In a multi-processor system, a plurality of processing units executes computer-executable instructions in parallel to enhance parallel processing capability of the computing device 100. The processing unit 110 can also be known as a central processing unit (CPU), microprocessor, controller and microcontroller.

The computing device 100 usually includes a plurality of computer storage mediums. Such mediums may be any attainable medium accessible by the computing device 100, including but not limited to, a volatile and non-volatile medium, a removable and non-removable medium. The memory 120 may be a volatile memory (e.g., a register, a cache, a Random Access Memory (RAM)), a non-volatile memory (such as, a Read-Only Memory (ROM), an Electrically Erasable Programmable Read-Only Memory (EEPROM), flash), or any combination thereof. The memory 120 may include a structure prediction module 122, which are configured to perform various functions described herein. The structure prediction module 122 may be accessed and operated by the processing unit 110 to implement the corresponding functions.

The storage device 130 may be a removable or non-removable medium, and may include a machine-readable medium (e.g., a memory, a flash drive, a magnetic disk) or any other medium, which may be used for storing information and/or data and be accessed within the computing device 100. The computing device 100 may further include additional removable/non-removable, volatile/non-volatile storage mediums. Although not shown in FIG. 1, there may be provided a disk drive for reading from or writing into a removable and non-volatile disk and an optical disc drive for reading from or writing into a removable and non-volatile optical disc. In such cases, each drive may be connected to a bus (not shown) via one or more data medium interfaces.

The communication unit 140 implements communication with another computing device via a communication medium. Additionally, functions of components of the computing device 100 may be implemented by a single computing cluster or a plurality of computing machines, and these computing machines may communicate through communication connections. Therefore, the computing device 100 may operate in a networked environment using a logic connection to one or more other servers, a Personal Computer (PC) or a further general network node.

The input device 150 may be one or more various input devices, such as a mouse, a keyboard, a trackball, a voice-input device, and the like. The output device 160 may be one or more output devices, e.g., a display, a loudspeaker, a printer, and so on. The computing device 100 may also communicate through the communication unit 140 with one or more external devices (not shown) as required, where the external device, e.g., a storage device, a display device, and so on, communicates with one or more devices that enable users to interact with the computing device 100, or with any device (such as a network card, a modem, and the like) that enable the computing device 100 to communicate with one or more other computing devices. Such communication may be executed via an Input/Output (I/O) interface (not shown).

In some implementations, apart from being integrated on an individual device, some or all of the respective components of the computing device 100 may also be set in the form of a cloud computing architecture. In the cloud computing architecture, these components may be remotely arranged and may cooperate to implement the functions described by the subject matter described herein. In some implementations, the cloud computing provides computation, software, data access and storage services without informing a terminal user of physical locations or configurations of systems or hardware providing such services. In various implementations, the cloud computing provides services via a Wide Area Network (such as Internet) using a suitable protocol. For example, the cloud computing provider provides, via the Wide Area Network, the applications, which can be accessed through a web browser or any other computing component. Software or components of the cloud computing architecture and corresponding data may be stored on a server at a remote location. The computing resources in the cloud computing environment may be consolidated at a remote datacenter or dispersed. The cloud computing infrastructure may provide, via a shared datacenter, the services even though they are shown as a single access point for the user. Therefore, components and functions described herein can be provided using the cloud computing architecture from a service provider at a remote location. Alternatively, components and functions may also be provided from a conventional server, or they may be mounted on a client device directly or in other ways.

The computing device 100 can be used for implementing protein structure prediction in various implementations of the subject matter described herein. In the implementations of the subject matter described herein, protein structure prediction is based on a plurality of constraints for structural properties of a protein to be predicted (referred to as a “target protein”). As shown in FIG. 1, the computing device 100 may receive, through the input device 150, a constraint set 170 for a structure of the target protein. The constraint set 170 may include a plurality of constraints for the structural properties of the target protein.

The computing device 100, for example, the structure prediction module 122 of the computing device 100, may perform prediction of the structure of the target protein based on the plurality of constraints and provides a prediction result 180 related to the structure of the target protein. The prediction result 180 indicates a spatial structure (e.g., a 3D spatial structure) of the target protein. For example, the prediction result 180 may include spatial coordination representations of main atoms in the target protein.

Although in the example shown in FIG. 1, the computing device 100 receives the constraint set 170 from the input device 150 and provides the prediction result 180 via the output device 160, this is merely illustrative without any limitation to the scope of the subject matter described herein. The computing device 100 may further receive the constraint set 170 from other devices (not shown) via the communication unit 140 and/or provide the prediction result 180 externally via the communication unit 140.

### Structural Properties and Spatial Coordinate Representation of Proteins

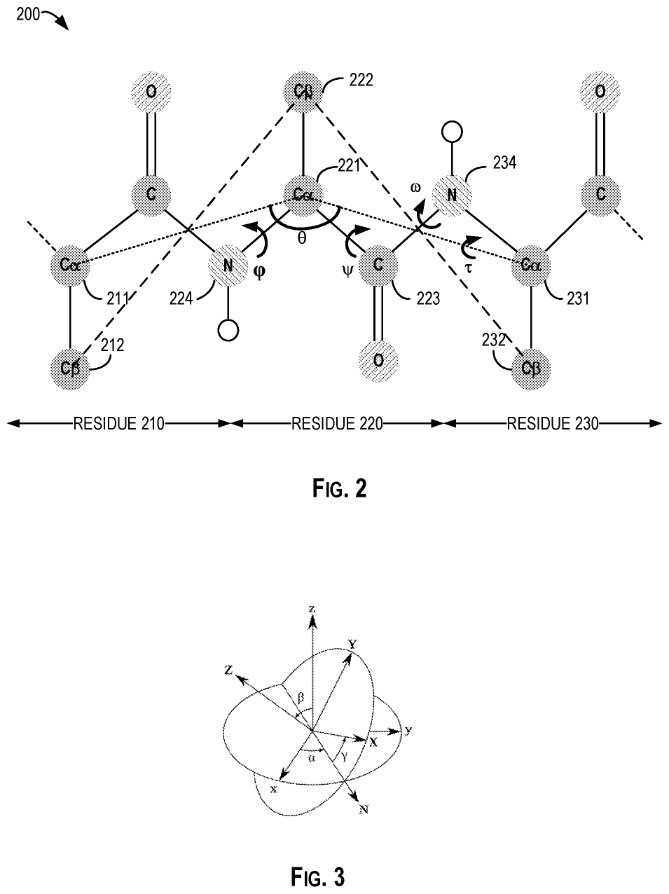

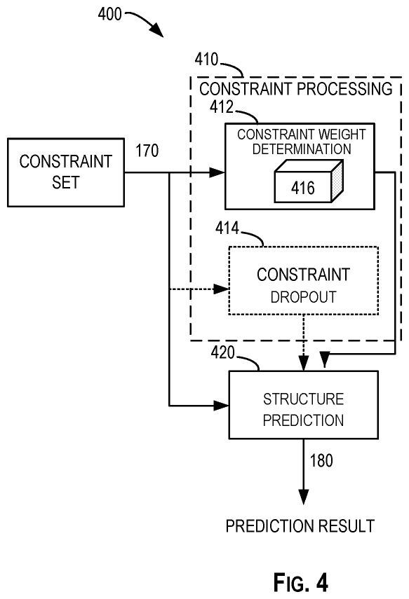

As mentioned above, the input required for protein structure prediction is constraint information for structural properties of a target protein, and the predicted structure can be represented by coordinates of atoms of the protein. To better understand the implementations of the subject matter described herein, reference is made to FIG. 2 and FIG. 3 to introduce structural properties and spatial coordinate representations of proteins, respectively.

FIG. 2 shows a structure of a fragment 200 of a protein which comprises a plurality of residues 210, 220, and 230. Each residue of the protein comprises N atoms, Cα atoms and C atoms on the main chain, as well as Cβ atoms and O atoms on side chains.

Structural properties of a protein may comprise inter-residue distances between a plurality of resides. Inter-residue distances may comprise distances between the same type of atoms in two resides, such as a Cα-Cα distance and a Cβ-Cβ distance. The Cα-Cα distance refers to a distance between pairwise Cα-Cα atoms (also referred to as an inter-residue Cα distance). The Cα-Cα distance may comprise a distance between a pair of neighboring Cα atoms or a distance between a pair of any non-neighboring Cα atoms, such as a distance between any two of Cα atoms 211, 221 and 231 in FIG. 2. The Cβ-Cβ distance refers to a distance between pairwise Cβ-Cβ atoms (also referred to as an inter-residue Cβ distance). The Cβ-Cβ distance may comprise a distance between a pair of neighboring Cβ atoms or a distance between a pair of any non-neighboring Cβ atoms, such as a distance between any two of Cβ atoms 212, 222 and 232 in FIG. 2.

The structural properties of the protein may further comprise inter-residue orientations between a plurality of resides. Inter-residue orientations may comprise an angle between a plurality of atoms in two resides, such as torsion angles φ and ω, backbone angles θ and τ, etc. as shown in FIG. 2. The torsion angle φ refers to a dihedral angle for an N—Cα chemical bond. The torsion angle ω refers to a dihedral angle for a chemical bond C—N. For examples, with respect to the residues 220 and 210, the torsion angle φ is a dihedral angle for a chemical bond between the N atom 224 and the Cα atom 221. With respect to the residues 220 and 230, the torsion angle ω is a dihedral angle for a chemical bond between the C atom 223 and the N atom 234. The backbone angle θ refers to a dihedral angle for a Cα-Cα-Cα chemical bond of neighboring residues. The backbone angle τ refers to a dihedral angle for a Cα-Cα chemical bond of neighboring residues. For example, for the residue 220, the backbone angle θ is the angle, at the Cα atom 221, of the triangle formed by its Cα atom 221 and the Cα atoms 211 and 231 in the neighboring residues 210 and 230, and the backbone angle τ is a dihedral angle of the line between the Cα atom 221 and the Cα atoms 231 (or 211).

The structural properties of the protein may further comprise other orientations between atoms of the protein. For example, the structural properties may further comprise a torsion angle ψ within a residue as shown in FIG. 2. The torsion angle ψ refers to a dihedral angle for a Cα-C chemical bond within a residue. For example, for the residue 220, the torsion angle ψ is a dihedral angle for a chemical bond between the Cα atom 221 and the C atom 223. In addition, the structural properties of the protein may further comprise bond lengths and bond angles between continuous atoms on the main chain. The bond lengths may comprise a bond length between N-Cα atoms, a bond length between Cα-C atoms, and a bond length between C—N atoms within each residue, etc. The bond angles may comprise bond angles between N-Cα-C atoms, between Cα-C—N atoms, and between C—N-Cα atoms within each residue, etc.

The 3D structure of the protein may be represented as a coordinate representation of each residue in the protein. To predict the structure of the protein, a spatial coordinate representation of the main atoms (e.g., the Cα atom or Cβ atom) of each residue in the protein may be determined. A spatial coordinate representation of a main atom may include coordinate parameters and orientation parameters for describing the spatial position of the main atom.

FIG. 3 illustrates an example spatial coordinate representation system 300 of an atom (the Cα atom or Cβ atom) of a protein. The spatial position of the atom may be represented through three coordinate parameters of Cartesian Coordinate System (x, y, z) in the spatial coordinate representation system 300. The orientation of the atom may be represented by three coordinate parameters (α, β, γ) of an Euler angle.

The Euler angle describes in the space, an angle obtained after a series of basic rotation from a known direction used for representing a certain fixed reference system (e.g., a coordinate system (x, y, z) shown in FIG. 3) to a new direction that represents another reference system (e.g., the coordinate system (X, Y, Z) in FIG. 3). A line of nodes (N) is a line where xy and XY coordinate planes intersect. In the three coordinate parameters (α, β, γ) of the Euler angle, a refers to an angle between the X-axis and N-axis, β refers to an angle between the z-axis and the Z-axis, and γ refers to an angle between the N-axis and the X-axis.

To predict the structure of the protein, if the spatial coordinate representation (e.g., parameters (x, y, z) and (α, β, γ)) of the Cα atom or Cβ atom of a residue is determined, the spatial coordinate representations of other atoms in the same residue, including the N atom, C atom, O atom and the other of Cα atom and Cβ atom, may also be determined respectively based on the spatial coordinate representation of the Cα atom or Cβ atom.

It should be appreciated that only one example of describing the spatial structure of the protein is presented. There may be other manners of representing the spatial structure, and the implementations of the subject matter described herein are not limited in this regard.

### Basic Work Principles

In the protein structure prediction, there are many techniques for determining predicted information of structural properties of a protein, e.g. the inter-residue distances and inter-residue orientations of the protein. The obtained predicted information is usually probability distribution information of a specific structural property within a certain range of property values. On the basis of the predicted information of the given structural properties of a protein, it is a more challenging task to effectively use the information to fold the 3D spatial structural (i.e., a tertiary structure) of the protein.

Some protein structure prediction models are proposed to predict the structure of the protein by using predicted information of a plurality structural properties of the protein as a plurality of constraints, to make the predicted structural properties satisfy those constraints. Usually, these structure prediction models directly take all constraints for the plurality of structural properties as input of the models, and treat all constraints equally during the structure prediction.

However, the predicted information for the structural properties of the protein may not be completely correct. For example, it is possible that only probability distribution information of a specific structural property within a certain range of property values can be obtained. Conflicts or redundancy might exist in the predicted information of respective structural properties or between the predicted information of different structural properties. In addition, since the inter-residue distances and inter-residue orientations depict the structure of the protein from different perspectives, this is prone to cause some of the information to be redundant in using for predicting the structure of the protein, and even cause conflicts.

A simple example is taken. For a triangle, its structure may be determined by one apex angle and two sides, which means that the remaining information is redundant for predicting the structure of the triangle. In addition, the redundant information might cause a conflict. For example, when two apex angles and two sides are given, the triangle formed by one of the apex angles and two sides might not conform to the other one of the given apex angles. Similar to the example regarding the triangle, during the protein structure prediction, the conflicts and redundancy of the predicted information that is not completely correct will affect the optimization of the protein structure. On one hand, a plurality of pieces of predicted information of the same residue that conflict with one another might push the optimization to a different direction. On the other hand, the conflicting and redundant predicted information between different residues might make the energy landscape of the target protein too rugged to efficiently optimize.

According to implementations of the subject matter described herein, an improved solution of protein structure prediction is proposed. According to the solution, a constraint set for a plurality of structural properties of a target protein is processed before it is used to perform prediction. Specifically, a plurality of weights corresponding to the plurality of constraints are determined respectively based on feature information of the plurality of constraints in the input constraint set. Each weight indicates a degree of influence of the corresponding constraint in prediction of a structure of the target protein. The structure of the target protein is predicted based on the plurality of constraints and the plurality of weights.

In this solution, pre-processing is performed on the constraints before using the constraint set to perform the prediction, and the weights determined for the plurality of constraints may decide a degree to which the constraints affect the prediction of the structure of the protein. For example, a constraint with a small weight may not be considered in predicting the structure of the protein, or it has a small influence on the optimization process of the structure. For a constraint with a large weight, it is desirable that the structural properties in the predicted structure of the protein can satisfy that constraint as much as possible. It is possible to solve potential conflicts in the constraint set and eliminate constraint redundancy through the pre-processing on the constraints for use. This enables accurate prediction of the structure of the target protein.

In some implementations, in addition to assigning the weights to process the constraints or as an alternative, the structure of the protein may be predicted in a plurality of iterations where in each iteration, a part of the constraints may be randomly discarded.

In some implementations, the prediction of the structure of the target protein is performed in an iterative optimization way. In some implementations, a good predicted structure generated in a previous iteration may be used to guide the prediction of the structure in next iteration. In one implementation, a good predicted structure generated in a previous iteration may be used to filter out a constraint(s) used in a next iteration from the constraint set, thereby implementing dynamic constraint filtration in an adaptive manner. In one implementation, a good predicted structure generated in a previous iteration may further be used to initialize a structure of the target protein to be optimized in the next iteration. As compared with randomly initializing the structure of the target protein in each iteration, “inheriting” a good predicted structure from a previous iteration to a next iteration may further improve the accuracy of the structure prediction.

Some example implementations of the subject matter described herein will be described in more detail below with reference to FIGS. 4-10.

### Example Architecture and Example Implementations of Constraint Processing

FIG. 4 illustrates a block diagram of a protein structure prediction system 400 according to some implementations of the subject matter described herein. The protein structure prediction system 400 may be implemented in a computing device 100, for example, included in the protein structure prediction module 122 of the computing device 100. In the example of FIG. 4, the system 400 includes a constraint processing module 410 and a structure prediction module 420. The system 400 is configured to determine the prediction result 180 related to the structure of the target protein based on the input constraint set 170 for the target protein.

The constraint set 170 includes a plurality of constraints for a plurality of structural properties of the target protein. The plurality of structural properties may include different types of structural properties of the target protein. In some implementations, the structural properties to be considered may include inter-residue distances and inter-residue orientations of a plurality of residues that form the target protein. For example, the inter-residue distances may include a distance between Cα-Cα atoms and/or a distance between Cβ-Cβ atoms of a pair of residues in the target protein. The inter-residue orientations may include angles between a plurality of atoms in pairwise residues in the target protein, such as the torsion angles φ and ω, the backbone angle θ, and the like. The structural properties may further include other properties between or within the residues of the target protein, for example, other distances or angles.

Each constraint in the constraint set 170 may indicate predicted information for a property value of a corresponding structural property. Since the target protein may consist of a plurality of residues, there may be a plurality of constraints for each structural property. For example, for the distance between Cβ-Cβ atoms, the constraint set 170 may include a distance between Cβ-Cβ atoms of a plurality of pairs of residues in the target protein. As another example, for each of the torsion angles φ and ω and the backbone angle θ, the constraint set 170 may also include a plurality of angles determined respectively for the plurality of pairs of residues. Generally, property values of structural properties may be predicted through various analysis techniques applied on the structural properties of the target protein. For example, the constraints in the constraint set 170 are determined based on sequence information and coevolution information sourced from Multiple Sequence Alignment (MSA) analysis. MSA refers to sequence alignments performed for more than three biological sequences of the protein, such as, a protein sequence, a DNA sequence or a RNA sequence. By using the structural property prediction techniques or solutions that are currently available or to be developed in the future, the generated predicted information may all be used in the constrain set to perform the protein structure prediction.

Depending on the used structural property prediction techniques, the predicted information indicated by one or more constraints in the constraint set 170 may not be accurate property values of the correspond structural properties, but may be probability distribution information of the property value of the structural properties. The probability distribution information may include probabilities of the property values in a property value range. As an example, regarding the distance between Cα-Cα atoms in two residues in a target residue, the corresponding probability distribution information may include probabilities of discrete distances within a distance range. For example, the distance range may be divided into 10 distance intervals, and the probability distribution information may include a probability of a distance interval being a ground-truth distance between the Cα-Cα atoms.

Upon the protein structure prediction, the constraints in the constraint set 170 are used to help constrain a structure of the target protein to be predicted, so that the structural properties of the structure can satisfy the constraints in the constraint set 170 as much as possible. As discussed above, since conflicts or redundancy between the constraints may exist in the obtained constraint set 170, it is desirable to pre-process these constraints before their use. The system of FIG. 4 includes the constraint processing module 410 to process the constraint set 170 to provide constraints to be used by the structure prediction module 420.

As shown in FIG. 4, the constraint processing module 410 includes a constraint weight determination module 412 configured to evaluate the quality of the constraints in the constraint set 170, so as to determine weights corresponding to the respective constraints. A weight is used to indicate a degree of influence of the corresponding constraint in prediction of a structure of the target protein. For example, each constraint may be assigned with a quality score within an interval from 0 to 1, where 1 indicates that the constraint is of the highest quality and may be assigned with a higher weight, while 0 indicates that the constraint is of the lowest quality and may be assigned with a lower weight or will not be selected to predict the structure of the target protein (for example, its weight is set to 0).

Upon determining the weights of the constraints, the constraint weight determination module 412 may extract feature information of the constraints in the constraint set 170. The constraint weight determination module 412 may determine, based on the extracted feature information, respective quality scores of the constraints by using a constraint quality analysis model 416. The quality scores of the constraints may be used to determine the weights of the constraints.

Generally, it is desirable to use a high-quality constraint for the structure prediction, where the high quality may be reflected in a way that the constraint is accurate, does not conflict with other constraints and is not redundant. The quality of the constraint may be reflected by the features of the constraint itself. For example, if a constraint indicates the probability distribution information of the property value of the corresponding structural property, a distribution shape corresponding to the probability distribution information may reflect, to a certain degree, whether the prediction of the property value is accurate. For example, the accurate prediction of the property value of the structural property generally has a sharp probability distribution with a prominent peak, while a poor prediction generally has a flat distribution with similar probabilities in respective intervals.

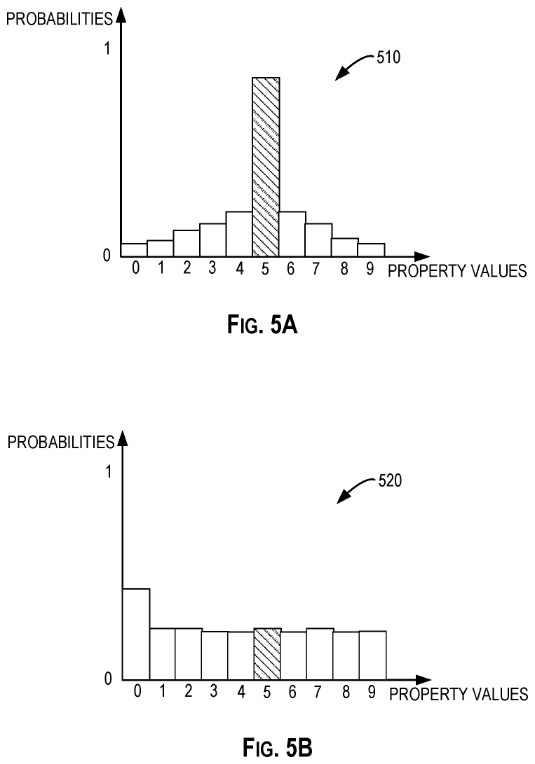

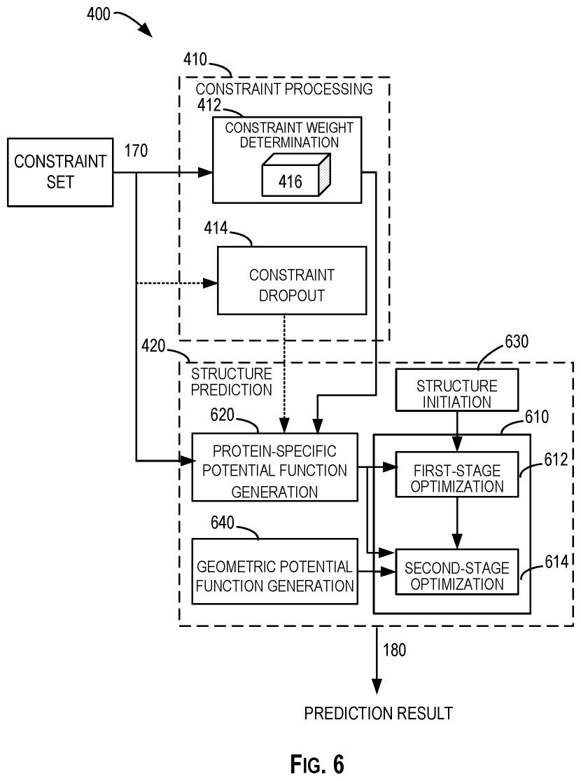

FIG. 5A and FIG. 5B illustrate two examples of constraints for a structural property. In these two examples, constraints are indicated by the probability distributions of property values of the structural property. The correct property value of the structural property is located at a property value interval corresponding to Bar No. 5 of the probability distribution. In the example of FIG. 5A, a probability distribution 510 indicated by the constraint has a significant peak, where the probability of Bin No. 5 is significantly higher than the probabilities of other bars. Therefore, if being applied in the protein structure prediction, the property value interval corresponding to Bin No. 5 is more likely used to affect the protein structure prediction. In the example of FIG. 5B, probabilities of respective bins of a probability distribution 520 are similar. The probability of Bin No. 0 is larger than the probabilities of other bins (including the probability of Bin No. 5), and thus the property value interval corresponding to Bin No. 0 is more likely used to affect the protein structure prediction. By comparing the examples of FIG. 5A and FIG. 5B, the probability distribution 510 may be considered to be of better quality.

In some implementations, upon extracting the feature information, the constraint weight determination module 412 may extract, from a constraint, features in one or more aspects that are capable of indicating the quality of that constraint. Of course, in an example in which the constraint is represented by the probability distribution information, the shape of the probability distribution is only a type of feature information that may represent the quality of the constraint. The feature information of other aspects of the constraint may also affect the quality of the constraint, and in turn affect the determination of its weight.

In some implementations, if a constraint in the constraint set 170 is indicated by the probability distribution information, the extracted feature information may include feature information related to the probability distribution, such as one or more of the following: a highest probability in the probability distribution; a median value of a bin having the highest probability in the probability distribution; a difference between the highest probability and a lowest probability in the probability distribution; a difference between the highest probability and a probability of its left neighboring bin; a difference between the highest probability and a probability of its right neighboring bin; a difference between the highest probability and the second highest probability; a difference between the median value of the bin having the highest probability and a median value of the bin having the second highest probability, and so on.

In some implementations, if a structural property indicated by a constraint is an inter-residue distance or an inter-residue orientation of a pair of residues in the protein, the feature information related to the pair of residues may also be extracted, which includes, for example, a sequential interval between the pair of residues on the secondary structure, a sequential interval normalized by the length of the target protein, and the like.

The constraint quality analysis model 416 may be defined as a machine learning models or a deep learning model (e.g., a neural network), configured to process the feature information extracted for each constraint in the constraint set 170. For each constraint, the extracted feature information may be combined together as an input to the constraint quality analysis model 416. An output of the constraint quality analysis model 416 is a quality score of the constraint, which may be, for example, a value between 0 and 1.

As an example, the constraint quality analysis model 416 may include a plurality of fully-connected (FC) layers that are sequentially connected, where each FC layer includes one or more processing nodes, and each processing node is configured as a corresponding activation function. For example, the first few FC layers may include a plurality of processing nodes whose activation functions may be selected as nonlinear activation functions, such as a ReLU function. The last FC layer may include a single processing node whose activation function may, for example, be selected as a sigmoid function to provide a normalized model output. It should be appreciated that one example structure of the constraint quality analysis model 416 is provided here. Other model structures are also possible.

In some implementations, the constraint quality analysis model 416 may be trained based on ground-truth property values of the plurality of structural properties in the known structures of proteins. Currently, ground-truth structures of a certain number of proteins have been determined in laboratories. These protein structures may be used as training data to train the constraint quality analysis model 416. For example, a CASP12 protein database provides a training set and a validation set available for model training. During the training of the constraint quality analysis model 416, a plurality of constraints (e.g., probability distribution information) of a plurality of structural properties of a protein with a known structure may be obtained, and quality scores may be labeled based on the ground-truth property values of the structural properties corresponding to the plurality of constraints.

The labeling for the constraints may follow some rules. If a constraint indicates the probability distribution information of the property value of the corresponding structural property, each property value interval in the probability distribution may be labeled. For example, for a bin greater than 20 Å (Angstrom) in the probability distribution information indicating an inter-residue distance, (1) if the native distance is greater than 20 Å in the bin and the probability of the bin in the probability distribution is greater than 0.9, the constraint is labeled with a quality score 1; (2) If the native distance is less than 20 Å and the probability of the bin in the probability distribution is greater than 0.9, the constraint is labeled with quality score 0; (3) if the probability of the bin in the probability distribution is less than 0.9, the bin is discarded, and the probabilities of other bins in the probability distribution are re-normalized. After the re-normalization, an expected value of the inter-residue distance is calculated based on there-normalized probability distribution. If the difference between the expected value and the ground-truth distance is greater than 10 Å, the constraint is labeled with a quality score of 0; otherwise the quality score of the constraint may be calculated based on the following:

$$1. - \frac{\vert E - G \vert}{10.} \qquad ( 1 )$$

where E represents the expected value of the probability distribution after the re-normalization, and G represents the native distance. Here, “native distance” refers to the ground-truth property value of the inter-residue distance, which may be determined from the known structure of the protein.

In the case that the constraints and the labeling of the constraints used in the training are determined, a model training technique may be leveraged to train the constraint quality analysis model 416 to enable it to learn how to determine the quality scores of the constraints based on the extracted feature information of the constraints. The specific model training technique used is not limited here.

The example implementation discussed above describes how the quality scores of the plurality of constraints in the constraint set 170 are determined by the constrain quality analysis model 416. The quality scores may be used to determine the weights of the plurality of constraints in constraint set 170. In some implementations, the quality scores or weights of one or more constraints in the constraint set 170 may also be indicated by the user manually.

The weights of the plurality of constraints are provided to the structure prediction module 420 to affect the prediction when the corresponding constraints are used to predict the structure of the target protein. The structure prediction module 420 uses a plurality of constraints in the constraint set 170 and determines a prediction result 180 of the structure of the target protein based on the weights of the used constraints.

In some implementations, to predict the structure of the target protein, the structure prediction model 420 may optimize the structure of the target protein through an iteration process. In each iteration, the structure prediction model 420 may generate at least one predicted structure of the target protein based on the constraints in the constraint set 170, and determine the target structure of the target protein based on the plurality of predicted structures generated in the plurality of iterations.

In an example implementation of the iterative optimization, the constraint processing module 410 may further include a constraint dropout module 414 which is configured to discard, during the iterative process for target protein prediction, partial constraints from all the constraints of the original constraint set 170 in each iteration, so as to obtain a reduced constraint set. In such implementation, the constraints used by the structure prediction model 420 in each iteration may not be the original constraint set 170, but the reduced constraint set.

Dropout is an operation that is often used in the training of deep neural network models to prevent the problem of over-fitting. The dropout operation refers to randomly making weights of processing nodes of some hidden layers in the network not work during the training, where the nodes that do not work may be temporarily considered not a part of the network structure, but the weights of these nodes are preserved (only not updated temporarily) so that these nodes can work again when inputting following samples.

In some implementations of the subject matter described herein, in the iterative process for optimization on the structure of the target protein, the protein may be predicted by using constraints in a different constraint subset in each iteration by randomly dropping out partial constraints, thereby easing or avoiding the conflicts of the constraints in the constraint set 170. In some implementations, a proportion of constraints dropped out in each iteration may be predetermined to be, such as, 30%, 20%, and the like. In some implementations, with respect to constraints for different types of structural properties in the constraint set 170, the constraint dropout module 414 may apply the dropout of constraints separately, so as to avoid conflicts of constraints from different aspects.

In some implementations, after a plurality of iterations, the structure prediction model 420 may determine a final target structure of the target protein from the predicted structures of the target protein generated by the last iteration. In some implementations, the structure prediction model 420 may use the constraints for different residues for the target protein in each iteration, and the constraint dropout module 414 discards constraints of other residues from the constraint set 170. As such, the predicted structure generated by the structure prediction module 420 in each iteration only represents a partial structure of the target protein, i.e., a folded structure of the residues with the constraints applied. After the plurality of iterations, the structure prediction module 420 may combine the folded structure determined for all the residues of the target protein in the plurality of iterations to obtain the final target structure of the target protein.

As mentioned above, the structure of the target protein may be indicated by the spatial coordinate representation of the main atoms, such as a Cα atom or Cβ atom, and the spatial coordinate representations of other atoms may be derived from the spatial coordinates of the Cα atom or Cβ atom. Therefore, the structure prediction module 420 may need to determine the spatial coordinate representation of the Cα atom or Cβ atom during the structure prediction. The structure prediction module 420 may first initialize the spatial coordinate representation of the Cα atom or Cβ atom, and iteratively optimize the spatial coordinate representation of the Cα atom or Cβ atom to make the final predicted structure conform to the used constraints. The structure prediction module 420 may perform the prediction through various protein structure prediction techniques.

Upon performing the structure prediction, the structure prediction module 420 may configure potential functions corresponding to the plurality of structural properties in the constraint set 170 (e.g., different types of inter-residue distances and different types of inter-residue angles) respectively, and optimize the structure of the target protein based on these potential functions. The potential functions created using the constraints of the structural properties of the target protein are specific to the target protein, and thus may also be referred to as “protein-specific potential functions”.

For example, if the constraint set 170 includes corresponding constraints for the distance between Cβ-Cβ atoms of neighboring residues, torsion angles φ and ω, and a backbone angle θ, the structure prediction module 420 may generate four protein-specific potential functions corresponding to these structural properties respectively. In each protein-specific potential function, a set of constraints for the corresponding structural properties of the target protein are weighted and combined, and the weight of each constraint is determined by the weight constraint determination module 412. For example, for the distance between the Cβ-Cβ atoms of the target protein, a protein-specific potential function may be generated using distances between a plurality of Cβ-Cβ atoms given in the constraint set 170. In the implementations of iterative optimization, the constraints used in each iteration may be different, and the corresponding potential functions may also be generated based on the used constraints and their weights.

In some implementations, the generation of the protein-specific potential functions is based on all the constraints of constraint set 170. In the implementation of the iterative optimization, for each iteration, the generation of the protein-specific potential functions may be based on the reduced constraint set after the constraint dropout module 414 performs dropout on the constraints in the constraint set 170.

The structure prediction module 420 may utilize any potential functions that are currently defined or to be defined in the future. In some implementations, if a constraint indicates the probability distribution information, the probability of the last bin in the probability distribution may be selected as a reference state. The structure prediction module 420 may calculate a log ratio value between the probability of each bin in the probability distributions and the reference state, and convert the log ratio value into continuous and differentiable potentials by cubic spline interpolation. In other implementations, the structure prediction module 420 may construct the potential functions in other ways.

After determining the protein-specific potential functions corresponding to the plurality of structural properties, the structure prediction module 420 may determine, based on the determined protein-specific potential functions, an objective function for the structure prediction model that is used in the protein structure prediction. The objective function may include a combination of the plurality of protein-specific potential functions, or their weighted combination. The weights of the protein-specific potential functions in the objective function may be considered as hyperparameters, and may be adjusted based on a reference protein data set (such as CASP12FM), which includes information of the reference proteins with known structures.

The structure prediction module 420 may be used to determine the structure of the target protein. The structure prediction model may be configured to determine the structure of the target protein by causing the objective function to reach a convergence target, so that the plurality of structural properties of the determined structure satisfy the constraints used in the protein-specific potential functions. The convergence target may be making the objective function minimize or reduce to an expected level. For example, the structure prediction model may be a gradient descent-based protein folding framework, which can reach the convergence target after multiple optimization steps.

### Example Implementations of Two-Stage Optimization of Protein Structure

The structure obtained from the optimization based on the protein-specific potential functions may conform to the constraints for the structural properties of the target protein in the constraint set 170. However, the inventors of the present application discovered that some structures generated based on such potential functions may not be reasonable biophysically, failing to conform to some basic geometry properties of proteins.

In some implementations, a two-stage optimization solution for the protein structure is proposed. In first-stage optimization, a plurality of intermediate predicted structures of the target protein are generated based on the protein-specific potential functions, and in second-stage optimization, the plurality of intermediate predicted structures obtained in the first stage are adjusted using geometric potential functions of proteins, to make a final result biophysically reasonable. The geometric potential function(s) used in the second stage is based on at least one constraint for a basic geometry of proteins.

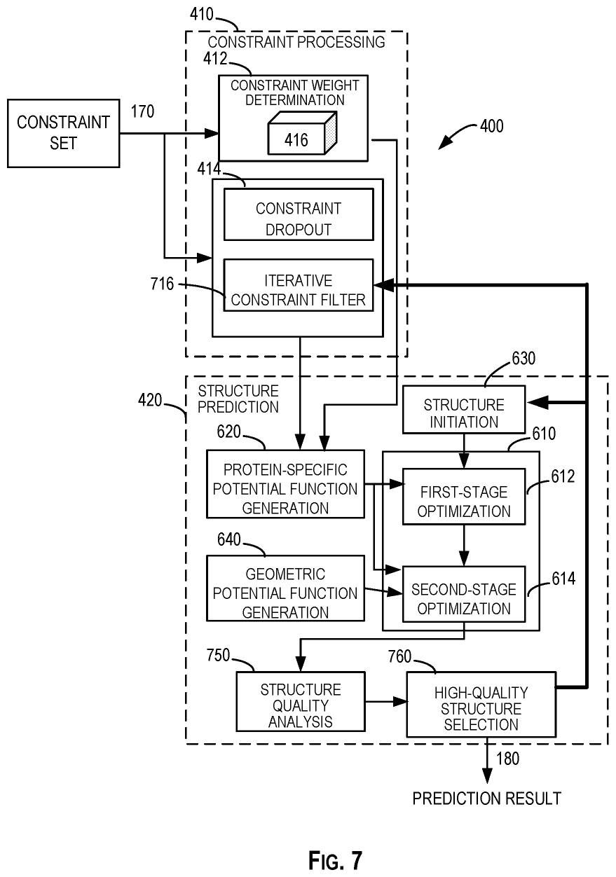

FIG. 6 illustrates a block diagram of a protein structure prediction system 400 according to some other implementations of the subject matter described herein. In the example of FIG. 6, the structure prediction module 420 is configured to perform a process of two-stage optimization on the protein structure.

As shown in FIG. 6, the structure prediction module 420 includes a two-stage optimization module 610 which includes a first-stage optimization module 612 and a second-stage optimization module 614. The two-stage optimization module 610 may further include a structure initialization module 630, which provides the first-stage optimization module 612 with one or more initial structures for use in the optimization. The structure prediction module 420 further includes a protein-specific potential function generation module 620 configured to generate a plurality of protein-specific potential functions corresponding to the plurality of structural properties based on the plurality of constraints in the constraint set 170 and their weights. The generation of the protein-specific potential functions has been described above, and will not be described in detail again here.

The structure prediction module 420 further includes a geometric potential function generation module 640 configured to generate one or more geometric potential functions to limit the geometry of the target protein, so that the predicted structure is a biophysically reasonable structure, and conforms to one or more constraints for basic geometry structural properties of proteins. The one or more constraints for the basic geometry structural properties of the proteins used herein are not specific to the target protein to be predicted, but satisfy general requirements for the geometry of proteins from the biophysical perspective.

In some implementations, in order to make the predicted protein structure more conform to the basic geometry structural properties, the basic geometry structural properties to be considered by the geometric potential function generation module 640 may include at least one of the following: a pairwise distance of two neighboring Cα atoms, a sequential interval between Cα atoms, a length of a peptide bond, a distance between an O atom within a residue and a N atom within a next residue, a distance between an O atom within a residue and a Cα atom within a next residue of the residue, and a difference of a distance between any pair of atoms (including the Cα atom, the Cβ atom, the N atom, the O atom and the C atom) and a sum of radiuses of the pair of atoms.

The geometric potential function generation module 640 may obtain property values of one or more basic geometry structural properties of a native peptide of a known protein, and use the obtained property values as constraints for these basic geometry structural properties. The geometric potential function generation module 640 may generate the geometric potential functions based on the constraints for the basic geometry structural properties.

In some implementations, the geometric potential function generation module 640 may generate at least one of a first geometric potential function to a sixth geometric potential function provided in following Equation (2) to Equation (7).

p₁=|d_{Cα}−3.8 Å| (2)

where p₁ represents a first geometric potential function, d_{Cα} represents a pairwise distance of two neighboring Cα atoms in a predicted structure of the target protein, and 3.8 Å is a statistical value of a pairwise distance of two neighboring Cα atoms determined from the native peptide.

p₂=(d_{Cα}−3.8 Å×(i−j)×1.1)⁴ (3)

where p₂ represents a second geometric potential function, (i−j) represents a sequential interval between Cα atoms in a predicted structure of the target protein.

p₃=(Lₚ−1.32 Å)² (4)

where p₃ represents a third geometric potential function, Lₚ represents a length of a peptide bond in a predicted structure of the target protein, and 1.32 Å is a statistical value of a length of a native peptide.

p₄=|d_{N-O}−2.8 Å| (5)

where p₄ represents a fourth geometric potential function, d_{N-O} represents a distance between an O atom within a residue and a N atom within a next residue in a predicted structure of the target protein, and 2.8 Å represents a statistical value of the distance between an O atom within a residue and a N atom within a next residue in the native peptide.

p₅=|d_{O-Cα}−2.69 Å| (6)

where p₅ represents a fifth geometric potential function, d_{O-Cα} represents a distance between an O atom within a residue and a Cα atom within a next residue of the residue in the predicted structure of the target protein, and 2.69 Å represents a statistical value of the distance between an O atom within a residue and a Cα atom within a next residue of the residue in the native peptide.

p₆=|d−(r₁+r₂)| (7)

where p₆ represents a sixth geometric potential function, d represents a difference of a distance between any pair of atoms (including the Cα atom, the Cβ atom, the N atom, the O atom and the C atom) in the predicted structure of the target protein, and r₁ and r₂ respectively represent a radius of the two atoms.

It should be appreciated that only some examples of geometric structural functions are presented above. In other implementations, more or less geometry structural properties may be considered, and more, less or different geometrical potential functions may be configured.

In the two-stage optimization module 610, the geometric potential functions are used for the second-stage optimization, and the protein-specific potential functions are used in both the first-stage optimization and the second-stage optimization. Specifically, the first-stage optimization module 612 generates one or more intermediate predicted structures of the target protein based on a plurality of protein-specific potential functions from the protein-specific potential function generation module 620. The structure prediction based on the plurality of protein-specific potential functions has been described above. The first-stage optimization module 612 may determine an objective function of the first-stage optimization (hereinafter referred to as “a first target function”) by combining the plurality of protein-specific potential functions, and determine one or more predicted structures of the target protein by causing the first objective function to reach a convergence target. The plurality of predicted structures facilitate may better sample the conformational space of the protein. The plurality of structural properties of the predicted structure generated in the first-stage optimization meet the constraints used in the plurality of protein-specific potential functions.

One or more optimized structures generated by the first-stage optimization module 612 are provided to the second-stage optimization module 614. The second-stage optimization module 614 may determine another objective function (hereinafter referred to as “a second target function”) based on one or more geometric potential functions from the geometric potential function generation module 640. The geometric potential function may, for example, include one or more of the first to the sixth geologic potential functions above. The second objective function may be determined, for example, by combining the geometric potential functions, so that when the second objective function reaches the convergence target (e.g., being minimized or reduced to an expected value), the basic geometry structural properties of one or more structures determined for the target protein all satisfy the constraints.

During the optimization, the second-stage optimization module 614 further takes the plurality of protein-specific potential functions into consideration so that the final structure still satisfies the one or more constraints in the constraint set 170. In the second-stage optimization, an initial structure to be optimized by the second-stage optimization module 614 is from one or more intermediate predicted structures of the first-stage optimization model 612. The second-stage optimization module 614 may use the structure prediction model to update at least one intermediate predicted structure by causing the first and second objective functions to reach their convergence targets.

Typically, in the first-stage optimization, the target protein has been rapidly folded from an initial structure, and the accuracy of the folded structure has been improved. An intermediate predicted structure determined after the first-stage optimization is substantially converged to satisfy the used constraints in the constraint set 170, but may not be reasonable in some local details. By means of the protein-specific potential functions and the geometric potential functions, the second-stage optimization may further fine-tune the local details, for example, to repair a broken peptide chain, correct some improprieties in the peptide, modify unreasonable secondary structures, adjust the overall structure, and the like.

In some implementations, the structures obtained by the second-stage optimization may be used to determine a prediction result 180 for the target protein. In some implementations, if the structure prediction module 420 performs an iterative optimization process, one or more intermediate predicted structures updated by the second-stage optimization module 614 in one iteration may be determined as the predicted structures generated for the target protein in this iteration, and may be provided to a next iteration.

### Example Implementation of Iterative Optimization and Iterative Constraint Filtering

In some implementations where the structure prediction module 420 performs the iterative optimization, good predicted structures generated in the previous iteration may be used to filter out, from the constraint set 170, constraints used in a next iteration, and/or may be used to initialize the structure of the target protein to be optimized in the next iteration. FIG. 7 illustrates such an implementation of the protein structure prediction system 400. A predicted structure provided from the previous iteration may be referred to as “decoy”.

In the example of FIG. 7, the constraint processing module 410 further includes an iterative constraint filter module 716, which is configured to select a good predicted structure from a plurality of predicted structures provided by the structure prediction module 410 in a previous iteration, and discard one or more constraints from the constraint set 170, to obtain a reduced constraint set for use in the current iteration. In each iteration, the constraints are discarded from the original constraint set 170.

The good predicted structure in the previous iteration may be used to help measure which constraints in the constraint sets 170 are poor constraints and which constraints are good constraints. In general, the most effective way to eliminate conflicts and redundancy in constraint set 170 is to compare the constraints in constraint set 170 with ground-truth values (i.e., ground-truth property values of the corresponding structural properties of the target protein). However, during the prediction process, such ground-truth values are unavailable. Generally, the structure prediction module 420 generates a plurality of predicted structures in each iteration to better sample the conformational space. In some implementations of the subject matter described herein, the good predicted structure in the previous iteration may be used to measure similar “ground-truth values” of the constraints.

In some implementations, the iterative constraint filter module 716 determines the property values of the plurality of structural properties from the selected one or more good predicted structures. For example, if the constraint set 170 includes one or more inter-residue distances and inter-residue orientations, the iterative constraint filter module 716 may correspondingly determine values of these inter-residue distances and inter-residue orientations in the predicted structure. For a structural property, the values determined from the plurality of predicted structures may be averaged or weighted averaged. The property value determined from the good predicted structure is used as a reference property value of the corresponding structural property.

For each of the plurality of structural properties or for some of the structural properties, the iterative constraint filter module 716 may compare the constraints for the corresponding structural property in the constraint set 170 with the corresponding reference values. If a difference between the property value indicated by a certain constraint of the plurality of constraints and the correspond reference property value is greater than a threshold difference, this constraint may be dropped out from the constraint set 170. The threshold difference has a predetermined value. For example, for a structural property related to a distance (e.g., the inter-residue distance), the threshold difference may be set to 9.0 Å; for a structural property related to an angle (e.g., the inter-residue angle), the threshold difference may be set to 9.0°. Certainly, these are merely some specific examples. Other threshold differences for the threshold or distance may also be set accordingly. In some implementations, different threshold differences may be set for different types of inter-residue distances and inter-residue angles.

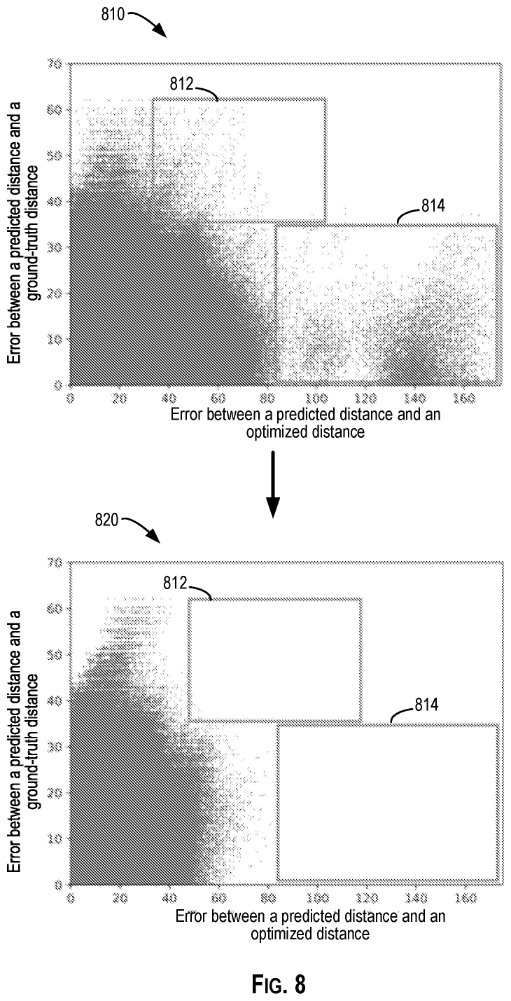

FIG. 8 illustrates a comparison of conflicts and redundancy between constraints in the constraint set before and after the iterative process. In FIG. 8, an example error map 810 shows an error of the protein in terms of the inter-residue distance. In FIG. 8, a horizontal axis indicates “an error between a predicted distance and an optimized distance”, where the predicted distance refers to the inter-residue distance in the constraint set of an example protein, and the optimized distance refers to an inter-residue distance of the best predicted structure shown by the system 400 in the first iteration (a statistical value in the case with a plurality of predicted structures). The vertical axis indicates “an error between a predicted distance and a ground-truth distance”, where the ground-truth distance refers to a ground-truth inter-residue distance determined from a known structure of a protein. Each point in example error map 810 indicates an error determined for a type of protein. In the example error map 810, a block 812 indicates that conflicts are present between the inter-residue distance in the constrain sets of some proteins and the inter-residue distance in the ground-truth structure, and a block 814 indicates that there are relatively large errors between the inter-residue distance in the constraint sets of some proteins and the inter-residue distance in the generated predicted structure.

After the constraint set of the protein is filtered in multiple iterations by using the better prediction result, the example error map 810 shows the error between the predicted distance and the optimized distance and the error between the predicted distance and the ground-truth distance included in the reduced constraint set obtained from the filtering. It can be seen that the errors corresponding to the blocks 812 and 814 in the error map 810 are removed, which means that the constraints having large errors and having conflicts with other constraints in the constraint set are removed.

It can be seen from the comparison of FIG. 8 that by iteratively filtering the constraints in the constraint set 170, it is possible to remove the constraints having conflicts and redundancy in a self-adaptive manner in the system 400. The predicted structure obtained after the multiple iterations may be determined depending on the reduced constraint set with less conflicts and redundancy. As such, the generated predicted structures may have high accuracy be higher. In some implementations, the number of iterations in the system 400 may be predetermined. In some implementations, after the last iteration is completed, the plurality of generated predicted structures may be used to determine a final prediction result 180 for the target protein. For example, a high-quality structure selection module 760 may select, from the plurality of prediction results in the last iteration, one or more prediction results as a final predicted structure(s) of the target protein.

In order to select good predicted structures (e.g., optimal decoys) from a plurality of predicted structures generated from each iteration, the structure prediction module 420 further includes a structure quality analysis model 750, which is configured to determine ranking of the plurality of predicted structures of the target protein generated in each iteration. The structure prediction module 420 further includes a high-quality structure selection module 760 configured to select one or more good predicted structures from the plurality of predicted structures in each iteration based on the ranking determined by the structure quality analysis model 750, to guide the optimization in a next iteration. For example, the high-quality structure selection module 760 may select one or more predicted structures ranked at higher places, or select one or more predicted structures ranked at places above a threshold.

Currently there have been some structure quality analysis models for proteins, used to measure quality of a predicted structure of a protein. Such structure analysis models are generally configured to evaluate the rationality of the predicted structure based on an overall potential energy of the protein, and indicate that the structure with the lowest potential energy has the highest quality. However, such structure analysis models highly depend on how the potential functions describe the native structure of the protein. In the example implementations of the subject matter described herein, instead of providing a definite quality score of a predicted structure by statistical potential energies, the structure quality analysis model 750 is configured to determine, based on a learning-to-rank algorithm, better or optimal ranking of the plurality of predicted structures of the target protein. Such a ranking order result may indicate relative-quality scores between the plurality of predicted structures.

In some implementations, the structure quality analysis model 750 includes a neural network model based on a learning-to-rank algorithm. In an implementation with the ranking-based algorithm, the structure quality analysis model 750 uses a learning-to-rank algorithm to perform a pairwise comparison of the predicted structures and determine the ranking of the plurality of predicted structures. In some implementations, the structure quality analysis model 750 may include one or more of a RankNET model and a LambDarank model to perform ranking of objects. In one implementation, the structure quality analysis model 750 may include a combined model of the RankNET model and the Lambdarange model. In the combined model, the inputs of the RankNet model and the LambDarank model are a pair of predicted structures, and the two models may determine a quality score for each of the predicted structures. As such, the ranking of the plurality of predicted structures may be determined based on the quality scores. The final ranking order in the plurality of predicted structures may be determined by jointly considering the rankings determined by the two models. For example, for each predicted structure, the ranking places provided by the two models may be averaged or weighted and averaged.

In some implementations with the combined model, the RankNet model and the LambDarank model may be configured with the same model structure, for example, including a scoring network consisting of four FC layers. The difference between the RankNet model and the LambDarank model is gradient calculation used in the two models during the model training. For example, the RankNet model may use the gradient calculation based on binary cross entropy, while the LambDarank model modifies the gradients of the RankNet model by multiplying the gradient by an absolute difference of normalized discounted cumulative gain (NDCG) of the two predicted structures to be ranked.

During the training of the RankNet model and the LambDarank model, a loss function of the two models may be determined by optimizing the ranking of the plurality of predicted structures, where the ranking is based on the quality scores output by the models for the plurality of predicted structures. Minimization of the loss functions are training objectives for the RankNet model and the LambDarank model. The creation of the loss functions for the RankNet model and the Lambdarange model will be briefly introduced below.

Assume that the probability P̅_{i,j} is defined as an average template modeling (TM) score according to the predicted structures i and j, the predicted structure i should be ranked before the predicted structure j. The probability P̅_{i,j} is calculated as follows:

P̅_{i,j}=½(1+Y_{i,j}), (8)

Y_{i,j}=max(−1,min(1,η*(yᵢ−yⱼ))) (9)

where yᵢ and yⱼ respectively represent the TM-scores of the two predicted structures i and y, and η is an adjustable parameter, and may be preset, for example, to 4, 3, 5 or any other value. The prediction probability may be determined by a sigmoid function, for example, as follows:

$${P}_{i , j} = \frac{1}{1 + {e}^{- σ ( {s}_{i} - {s}_{j} )}} \qquad ( 10 )$$

where Sᵢ and Sⱼ respectively represent the predicted quality scores provided by the RankNet model or the LambDarank model, and σ is an adjustable parameter, and may, for example, be preset to 1 or any other value.

The loss function for example may be determined as follows based on binary cross entropy:

Loss=ΣₜΣ_{i,j,i≠j}−P̅_{i,j}logP_{i,j}−(1−P_{i,j})log(1−P_{i,j}) (11)

where t represents an index of the protein used in the training. In some implementations, the training data of the RankNet model or the Lambdarange model may be based on the structures of the known proteins.

Based on the loss function in Equation (11), the gradient for use in the training of the RankNet model, e.g., the gradient with respect to the direction wₖ, is calculated as follows:

$$\frac{\partial Loss}{\partial {w}_{k}} = σ ( \frac{1}{2} ( 1 - {Y}_{i , j} ) - \frac{1}{1 + {e}^{- σ ( ) {s}_{i} - {s}_{j} )}} ) ( \frac{\partial {S}_{i}}{\partial {w}_{k}} - \frac{\partial {S}_{j}}{\partial {w}_{k}} ) = {λ}_{i , j} ( \frac{\partial {S}_{i}}{\partial {w}_{k}} - \frac{\partial {S}_{j}}{\partial {w}_{k}} ) . \qquad ( 12 )$$

The LambdaRank model further modifies the parameter λ_{i,j} in Equation (12) by following Equation (13), based on NDCG of the predicted structures:

$${λ}_{i , j} = σ ( \frac{1}{2} ( 1 - {Y}_{i , j} ) - \frac{1}{1 + {e}^{- σ ( {s}_{i} - {s}_{j} )}} ) \vert {Δ}_{NDCG} \vert \qquad ( 13 )$$

where |Δ_{NDCG}| indicates an absolute difference determined for the predicted structures i and j after switching the order of the predicted structures i and j.

It has been discussed above the ranking of the plurality of predicted structures in one iteration by combining two types of different neural network models. In some implementations, the structure quality analysis model 750 may also use only one type of neural network model such as a RankNet model or a Lambdarange model, or any other type of neural network model.

In some implementations, in addition to being used for iterative filtering of the constraints in the constraint set 170, or as an alternative, one or more good predicted structures generated in the previous iteration may also be used to determine the initial structure of the target protein to be used in a next iteration. As shown in FIG. 7, one or more predicted structures selected by the high-quality structure selection module 760 are provided to the structure initialization module 630. These predicted structures are used as template structures. In some implementations, the structure initialization module 630 may apply random perturbation data to the obtained one or more predicted structures, and provide the perturbed predicted structures as initial structures for the following structure optimization module, i.e., the first-stage optimization module 612. In some examples, a predicted structure may be indicated by the spatial coordinate representation of the Cα atom or Cβ atom of the target protein. In this case, the structure initialization module 630 may apply the perturbation data by randomly modifying spatial coordinate representations of these atoms (e.g., modifying one or more parameter values in the spatial coordinates representations). In some examples, the structure initialization module 630 may select a random value from a Gaussian distribution to modify the spatial coordinate representation of the Cα atom or Cβ atom. Other methods of generating random values are also possible.

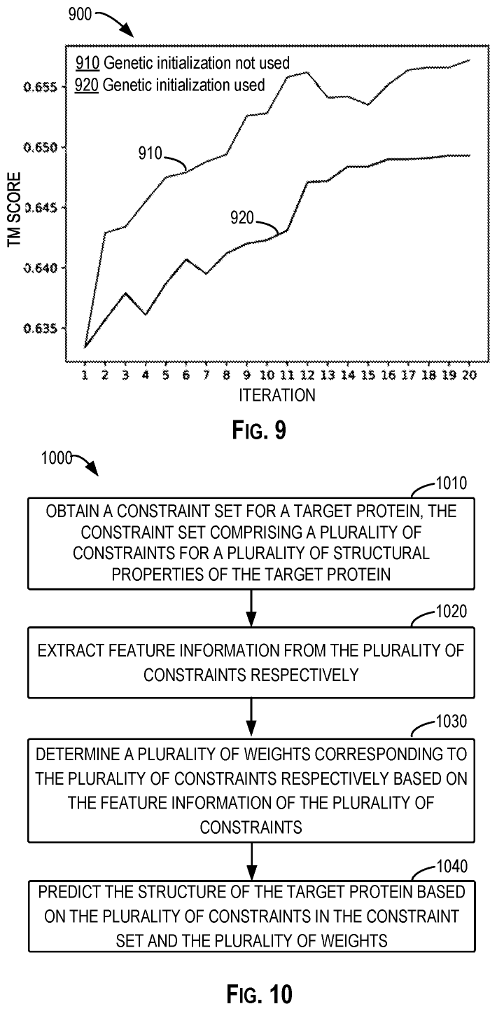

By using the predicted structures of the previous iteration to perform structural initialization of next iteration, the previously obtained prediction results may be inherited. Such initialization may also be referred to as “genetic initialization”. The genetic initialization may enable a more accurate prediction result 180 of the target protein 180. FIG. 9 illustrates an example comparison map 900 of iterative protein structure predictions with and without genetic initialization. During the iterative prediction without the genetic initialization, the initial structure in each iteration is a random structure determined by random initialization.

In FIG. 9, a curve 910 indicates TM scores of predicted structures from different iterations without genetic initialization, and a curve 920 indicates TM scores of predicted structures from different iterations with genetic initialization. A TM score can be used to measure accuracy of a structure of a protein. It can be seen by the comparison of the two curves that starting from the second iteration, the accuracy of the predicted structures generated based on genetic initialization is always higher than the predicted structures generated based on the random initialization only.

### Example Process

FIG. 10 illustrates a flowchart of a process 1000 of protein structure prediction in accordance with some implementations of the subject matter described herein. The process 1000 can be implemented by the computing device 100.

At block 1010, the computing device 100 obtains a constraint set for a target protein, the constraint set comprising a plurality of constraints for a plurality of structural properties of the target protein. At block 1020, the computing device 100 extracts feature information from the plurality of constraints respectively. At block 1030, the computing device 100 determine a plurality of weights corresponding to the plurality of constraints respectively based on the feature information of the plurality of constraints. Each weight indicates a degree of influence of the corresponding constraint in prediction of a structure of the target protein. At block 1040, the computing device 100 predicts the structure of the target protein based on the plurality of constraints in the constraint set and the plurality of weights.

In some implementations, the plurality of structural properties comprise inter-residue distances and inter-residue orientations of a plurality of residues that form the target protein. In some implementations, the plurality of constraints indicate probability distribution information of property values for the plurality of structural properties.

In some implementations, determining the plurality of weights corresponding to the plurality of constraints respectively comprises: determining, based on the extracted feature information, a plurality of quality scores for the plurality of constraints respectively using a constraint quality analysis model, the constraint quality analysis model being trained with ground-truth property values of a plurality of structural properties in a known structure of a protein; and assigning the plurality of weights to the plurality of constraints based on the plurality of quality scores for the plurality of constraints.

In some implementations, predicting the structure of the target protein comprises: predicting the structure of the target protein in a plurality of iterations, in each iteration, discarding at least one constraint from the constraint set, to obtain a reduced constraint set, and generating at least one predicted structure of the target protein based on the reduced constraint set and the weights assigned to a plurality of constraints in the reduced constraint set; and determining the structure of the target protein based on a plurality of predicted structures generated in the plurality of iterations.

In some implementations, predicting the structure of the target protein comprises: generating a plurality of protein-specific potential functions corresponding to the plurality of structural properties respectively, each protein-specific potential function being based on weighting of a group of constraints for the corresponding structural property in the constraint set, and the weighting being based on respective weights for the group of constraints; determining, based on the plurality of protein-specific potential functions, a first objective function for a structure prediction model used for predicting a structure of protein; and determining the structure of the target protein using the structure prediction model by at least causing the first objective function to reach a convergence target, the plurality of structural properties of the structure satisfying the constraints used in the plurality of protein-specific potential functions.

In some implementations, determining the structure of the target protein by at least causing the first objective function to reach the convergence target comprises: generating at least one geometric potential function, the at least one geometric potential function being based on at least one constraint for at least one basic geometry structural property of a protein, and the at least one constraint being based on a property value of the at least one basic geometry structural property determined from a native peptide of a known protein; determining a second objective function for the structure prediction model based on the at least one geometric potential function; determining the structure of the target protein using the structure prediction model by causing the first and second objective functions to reach their convergence targets respectively, the plurality of structural properties of the structure satisfying the constraints used in the plurality of protein-specific potential functions, and a geometry of the structure satisfying the constraint used in the at least one geometric potential function.

In some implementations, determining the structure of the target protein using the structure prediction model by causing the first and second objective functions to reach their convergence targets respectively comprises: in a first stage, determining at least one intermediate predicted structure of the target protein by causing the first objective function to reach the convergence target, the plurality of structural properties of the at least one intermediate predicted structure satisfying the constraints used in the plurality of protein-specific potential functions; and in a second stage, updating the at least one intermediate predicted structure by causing the first and second objective functions to reach their convergence targets, to determine the structure of the target protein.

In some implementations, the at least one basic geometry structural property comprises at least one of the following: a pairwise distance of two neighboring Cα atoms, a sequential interval between Cα atoms, a length of a peptide bond, a distance between an O atom within a residue and a N atom within a next residue, a distance between an O atom within a residue and a Cα atom within a next residue of the residue, and a difference of a distance between any pair of atoms and a sum of radiuses of the pair of atoms.

In some implementations, predicting the structure of the target protein comprises: predicting the structure of the target protein in a plurality of iterations, in a given iteration of the plurality of iterations, selecting at least one of a plurality of predicted structures generated in a previous iteration of the given iteration, determining, from the at least one selected predicted structure, a plurality of reference property values for the plurality of structural properties, and determining respective differences between the plurality of constraints for the plurality of structural properties in the constrain set and the plurality of determined reference property values, and in accordance with a determination that the difference between a property value indicated by at least one of the plurality of constraints and the corresponding reference property value exceeds a threshold difference, discarding the at least one constraint from the constraint set, to obtain a reduced constraint set, and
determining a plurality of predicted structures of the target protein in the given iteration based on the reduced constraint set and the weights assigned to the constraints in the reduced constraint set.

In some implementations, determining a plurality of predicted structures of the target protein in the given iteration comprises: in the given iteration, determining at least one initial structure of the target protein based on the at least one selected predicted structure; and determining the plurality of predicted structures of the target protein in the given iteration by optimizing the at least one initial structure.

In some implementations, selecting the at least one predicted structure comprises: determining ranking of the plurality of predicted structures generated in the previous iteration using a structure quality analysis model, the structure quality analysis model comprising one or more neural network models based on ranking learning; and selecting the at least one predicted structure from the plurality of predicted structures based on the ranking.

### Example Implementations

Some example implementations of the subject matter described herein are listed below.

In an aspect, the subject matter described herein provides a computer-implemented method. The method comprises: obtaining a constraint set for a target protein, the constraint set comprising a plurality of constraints for a plurality of structural properties of the target protein; extracting feature information from the plurality of constraints respectively; determining a plurality of weights corresponding to the plurality of constraints respectively based on the feature information of the plurality of constraints, each weight indicating a degree of influence of the corresponding constraint in prediction of a structure of the target protein; and predicting the structure of the target protein based on the plurality of constraints in the constraint set and the plurality of weights.

In another aspect, the subject matter described herein provides an electronic device. The electronic device comprises: a processor; and a memory coupled to the processor and having instructions stored thereon which, when executed by the processor, cause the device to perform the following acts: obtaining a constraint set for a target protein, the constraint set comprising a plurality of constraints for a plurality of structural properties of the target protein; extracting feature information from the plurality of constraints respectively; determining a plurality of weights corresponding to the plurality of constraints respectively based on the feature information of the plurality of constraints, each weight indicating a degree of influence of the corresponding constraint in prediction of a structure of the target protein; and predicting the structure of the target protein based on the plurality of constraints in the constraint set and the plurality of weights.

In some implementations, the plurality of structural properties comprise inter-residue distances and inter-residue orientations of a plurality of residues that form the target protein. In some implementations, the plurality of constraints indicate probability distribution information of property values for the plurality of structural properties.

In some implementations, determining the plurality of weights corresponding to the plurality of constraints respectively comprises: determining, based on the extracted feature information, a plurality of quality scores for the plurality of constraints respectively using a constraint quality analysis model, the constraint quality analysis model being trained with ground-truth property values of a plurality of structural properties in a known structure of a protein; and assigning the plurality of weights to the plurality of constraints based on the plurality of quality scores for the plurality of constraints.

In some implementations, predicting the structure of the target protein comprises: predicting the structure of the target protein in a plurality of iterations, in each iteration, discarding at least one constraint from the constraint set, to obtain a reduced constraint set, and generating at least one predicted structure of the target protein based on the reduced constraint set and the weights assigned to a plurality of constraints in the reduced constraint set; and determining the structure of the target protein based on a plurality of predicted structures generated in the plurality of iterations.

In some implementations, predicting the structure of the target protein comprises: generating a plurality of protein-specific potential functions corresponding to the plurality of structural properties respectively, each protein-specific potential function being based on weighting of a group of constraints for the corresponding structural property in the constraint set, and the weighting being based on respective weights for the group of constraints; determining, based on the plurality of protein-specific potential functions, a first objective function for a structure prediction model used for predicting a structure of protein; and determining the structure of the target protein using the structure prediction model by at least causing the first objective function to reach a convergence target, the plurality of structural properties of the structure satisfying the constraints used in the plurality of protein-specific potential functions.

In some implementations, determining the structure of the target protein by at least causing the first objective function to reach the convergence target comprises: generating at least one geometric potential function, the at least one geometric potential function being based on at least one constraint for at least one basic geometry structural property of a protein, and the at least one constraint being based on a property value of the at least one basic geometry structural property determined from a native peptide of a known protein; determining a second objective function for the structure prediction model based on the at least one geometric potential function; determining the structure of the target protein using the structure prediction model by causing the first and second objective functions to reach their convergence targets respectively, the plurality of structural properties of the structure satisfying the constraints used in the plurality of protein-specific potential functions, and a geometry of the structure satisfying the constraint used in the at least one geometric potential function.

In some implementations, determining the structure of the target protein using the structure prediction model by causing the first and second objective functions to reach their convergence targets respectively comprises: in a first stage, determining at least one intermediate predicted structure of the target protein by causing the first objective function to reach the convergence target, the plurality of structural properties of the at least one intermediate predicted structure satisfying the constraints used in the plurality of protein-specific potential functions; and in a second stage, updating the at least one intermediate predicted structure by causing the first and second objective functions to reach their convergence targets, to determine the structure of the target protein.

In some implementations, the at least one basic geometry structural property comprises at least one of the following: a pairwise distance of two neighboring Cα atoms, a sequential interval between Cα atoms, a length of a peptide bond, a distance between an O atom within a residue and a N atom within a next residue, a distance between an O atom within a residue and a Cα atom within a next residue of the residue, and a difference of a distance between any pair of atoms and a sum of radiuses of the pair of atoms.

In some implementations, predicting the structure of the target protein comprises: predicting the structure of the target protein in a plurality of iterations, in a given iteration of the plurality of iterations, selecting at least one of a plurality of predicted structures generated in a previous iteration of the given iteration, determining, from the at least one selected predicted structure, a plurality of reference property values for the plurality of structural properties, and determining respective differences between the plurality of constraints for the plurality of structural properties in the constrain set and the plurality of determined reference property values, and in accordance with a determination that the difference between a property value indicated by at least one of the plurality of constraints and the corresponding reference property value exceeds a threshold difference, discarding the at least one constraint from the constraint set, to obtain a reduced constraint set, and
determining a plurality of predicted structures of the target protein in the given iteration based on the reduced constraint set and the weights assigned to the constraints in the reduced constraint set.

In some implementations, determining a plurality of predicted structures of the target protein in the given iteration comprises: in the given iteration, determining at least one initial structure of the target protein based on the at least one selected predicted structure; and determining the plurality of predicted structures of the target protein in the given iteration by optimizing the at least one initial structure.

In some implementations, selecting the at least one predicted structure comprises: determining ranking of the plurality of predicted structures generated in the previous iteration using a structure quality analysis model, the structure quality analysis model comprising one or more neural network models based on ranking learning; and selecting the at least one predicted structure from the plurality of predicted structures based on the ranking.

In a further aspect, the subject matter described herein provides a computer program product being tangibly stored in a non-transitory computer storage medium and comprising machine-executable instructions, the machine-executable instructions, when executed by a device, causing the device to perform one or more implementations of the above method.

In a further aspect, the subject matter described herein provides a computer readable medium having machine-executable instructions stored thereon, the machine-executable instructions, when executed by a device, causing the device to perform the method according to the above aspect.

The functionalities described herein can be performed, at least in part, by one or more hardware logic components. As an example, and without limitation, illustrative types of hardware logic components that can be used include field-programmable gate arrays (FPGAs), Application-specific Integrated Circuits (ASICs), application-specific standard products (ASSPs), system-on-a-chip systems (SOCs), complex programmable logic devices (CPLDs), and the like.

Program code for carrying out the methods of the subject matter described herein may be written in any combination of one or more programming languages. The program code may be provided to a processor or controller of a general-purpose computer, special purpose computer, or other programmable data processing apparatus such that the program code, when executed by the processor or controller, causes the functions/operations specified in the flowcharts and/or block diagrams to be implemented. The program code may be executed entirely or partly on a machine, executed as a stand-alone software package partly on the machine, partly on a remote machine, or entirely on the remote machine or server.

In the context of this disclosure, a machine-readable medium may be any tangible medium that may contain or store a program for use by or in connection with an instruction execution system, apparatus, or device. The machine-readable medium may be a machine-readable signal medium or a machine-readable storage medium. A machine-readable medium may include but is not limited to an electronic, magnetic, optical, electromagnetic, infrared, or semiconductor system, apparatus, or device, or any suitable combination of the foregoing. More specific examples of the machine-readable storage medium would include an electrical connection having one or more wires, a portable computer diskette, a hard disk, a random-access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Further, while operations are depicted in a particular order, this should not be understood as requiring that such operations are performed in the particular order shown or in sequential order, or that all illustrated operations are performed to achieve the desired results. In certain circumstances, multitasking and parallel processing may be advantageous. Likewise, while several specific implementation details are contained in the above discussions, these should not be construed as limitations on the scope of the subject matter described herein, but rather as descriptions of features that may be specific to particular implementations. Certain features that are described in the context of separate implementations may also be implemented in combination in a single implementation. Rather, various features described in a single implementation may also be implemented in multiple implementations separately or in any suitable sub-combination.

Although the subject matter has been described in language specific to structural features and/or methodological acts, it is to be understood that the subject matter specified in the appended claims is not necessarily limited to the specific features or acts described above. Rather, the specific features and acts described above are disclosed as example forms of implementing the claims.

1. A computer-implemented method, comprising:
obtaining a constraint set for a target protein, the constraint set comprising a plurality of constraints for a plurality of structural properties of the target protein;
extracting feature information from the plurality of constraints respectively;
determining a plurality of weights corresponding to the plurality of constraints respectively based on the feature information of the plurality of constraints, each weight indicating a degree of influence of the corresponding constraint in prediction of a structure of the target protein; and
predicting the structure of the target protein based on the plurality of constraints in the constraint set and the plurality of weights.

2. The method of claim 1, wherein the plurality of structural properties comprise inter-residue distances and inter-residue orientations of a plurality of residues that form the target protein; and
wherein the plurality of constraints indicate probability distribution information of property values for the plurality of structural properties.

3. The method of claim 1, wherein determining the plurality of weights corresponding to the plurality of constraints respectively comprises:
determining, based on the extracted feature information, a plurality of quality scores for the plurality of constraints respectively using a constraint quality analysis model, the constraint quality analysis model being trained with ground-truth property values of a plurality of structural properties in a known structure of a protein; and
assigning the plurality of weights to the plurality of constraints based on the plurality of quality scores for the plurality of constraints.

4. The method of claim 1, wherein predicting the structure of the target protein comprises:
predicting the structure of the target protein in a plurality of iterations, in each iteration,
discarding at least one constraint from the constraint set, to obtain a reduced constraint set, and
generating at least one predicted structure of the target protein based on the reduced constraint set and the weights assigned to a plurality of constraints in the reduced constraint set; and
determining the structure of the target protein based on a plurality of predicted structures generated in the plurality of iterations.

5. The method of claim 1, wherein predicting the structure of the target protein comprises:
generating a plurality of protein-specific potential functions corresponding to the plurality of structural properties respectively, each protein-specific potential function being based on weighting of a group of constraints for the corresponding structural property in the constraint set, and the weighting being based on respective weights for the group of constraints;
determining, based on the plurality of protein-specific potential functions, a first objective function for a structure prediction model used for predicting a structure of protein; and
determining the structure of the target protein using the structure prediction model by at least causing the first objective function to reach a convergence target, the plurality of structural properties of the structure satisfying the constraints used in the plurality of protein-specific potential functions.

6. The method of claim 5, wherein determining the structure of the target protein by at least causing the first objective function to reach the convergence target comprises:
generating at least one geometric potential function, the at least one geometric potential function being based on at least one constraint for at least one basic geometry structural property of a protein, and the at least one constraint being based on a property value of the at least one basic geometry structural property determined from a native peptide of a known protein;
determining a second objective function for the structure prediction model based on the at least one geometric potential function;
determining the structure of the target protein using the structure prediction model by causing the first and second objective functions to reach their convergence targets respectively, the plurality of structural properties of the structure satisfying the constraints used in the plurality of protein-specific potential functions, and a geometry of the structure satisfying the constraint used in the at least one geometric potential function.

7. The method of claim 6, wherein determining the structure of the target protein using the structure prediction model by causing the first and second objective functions to reach their convergence targets respectively comprises:
in a first stage, determining at least one intermediate predicted structure of the target protein by causing the first objective function to reach the convergence target, the plurality of structural properties of the at least one intermediate predicted structure satisfying the constraints used in the plurality of protein-specific potential functions; and
in a second stage, updating the at least one intermediate predicted structure by causing the first and second objective functions to reach their convergence targets, to determine the structure of the target protein.

8. The method of claim 7, wherein the at least one basic geometry structural property comprises at least one of the following:
a pairwise distance of two neighboring Cα atoms,
a sequential interval between Cα atoms,
a length of a peptide bond,
a distance between an O atom within a residue and a N atom within a next residue,
a distance between an O atom within a residue and a Cα atom within a next residue of the residue, and
a difference of a distance between any pair of atoms and a sum of radiuses of the pair of atoms.

9. The method of claim 1, wherein predicting the structure of the target protein comprises:
predicting the structure of the target protein in a plurality of iterations, in a given iteration of the plurality of iterations,
selecting at least one of a plurality of predicted structures generated in a previous iteration of the given iteration,
determining, from the at least one selected predicted structure, a plurality of reference property values for the plurality of structural properties, and
determining respective differences between the plurality of constraints for the plurality of structural properties in the constrain set and the plurality of determined reference property values, and
in accordance with a determination that the difference between a property value indicated by at least one of the plurality of constraints and the corresponding reference property value exceeds a threshold difference, discarding the at least one constraint from the constraint set, to obtain a reduced constraint set, and
determining a plurality of predicted structures of the target protein in the given iteration based on the reduced constraint set and the weights assigned to the constraints in the reduced constraint set.

10. The method of claim 9, wherein determining a plurality of predicted structures of the target protein in the given iteration comprises: in the given iteration,
determining at least one initial structure of the target protein based on the at least one selected predicted structure; and
determining the plurality of predicted structures of the target protein in the given iteration by optimizing the at least one initial structure.

11. The method of claim 9, wherein selecting the at least one predicted structure comprises:
determining ranking of the plurality of predicted structures generated in the previous iteration using a structure quality analysis model, the structure quality analysis model comprising one or more neural network models based on ranking learning; and
selecting the at least one predicted structure from the plurality of predicted structures based on the ranking.

12. An electronic device, comprising:
a processing unit; and
a memory coupled to the processing unit and having instructions stored thereon, the instructions, when executed by the processing unit, causing the device to perform acts of:
obtaining a constraint set for a target protein, the constraint set comprising a plurality of constraints for a plurality of structural properties of the target protein;
extracting feature information from the plurality of constraints respectively;
determining a plurality of weights corresponding to the plurality of constraints respectively based on the feature information of the plurality of constraints, each weight indicating a degree of influence of the corresponding constraint in prediction of a structure of the target protein; and
predicting the structure of the target protein based on the plurality of constraints in the constraint set and the plurality of weights.

13. The device of claim 12, wherein determining the plurality of weights corresponding to the plurality of constraints respectively comprises:
determining, based on the extracted feature information, a plurality of quality scores for the plurality of constraints respectively using a constraint quality analysis model, the constraint quality analysis model being trained with ground-truth property values of a plurality of structural properties in a known structure of a protein; and
assigning the plurality of weights to the plurality of constraints based on the plurality of quality scores for the plurality of constraints.

14. The device of claim 12, wherein predicting the structure of the target protein comprises:
predicting the structure of the target protein in a plurality of iterations, in each iteration,
discarding at least one constraint from the constraint set, to obtain a reduced constraint set, and
generating at least one predicted structure of the target protein based on the reduced constraint set and the weights assigned to a plurality of constraints in the reduced constraint set; and
determining the structure of the target protein based on a plurality of predicted structures generated in the plurality of iterations.

15. A computer program product being tangibly stored in a computer storage medium and comprising machine-executable instructions which, when executed by a device, cause the device to perform the method of claim 1.
